SEQUENCE LISTING (4 sequences)

SEQ ID NO: 1 - protein, 325 aa, Pseudomonas diminuta
MQTRRVVLKSAAARTLLGGLAGCATWLDRSAQAMRSIRARPITISEAGFTLTHEDISAARQDSCVLGQSSSVAQSSSGKGCERIARQSGWRANDCRCVDFRYRSRRQFIGRGFAGCRRSYLAATGLWFDPPLSMRLRYVEELTLVLPAVRFNMASKYTGIRAGIIKVATTGKATPFQELVLKAAARASLATGVPVTTHTAASQRDGERGRPPFLSPKLEPSRVCIGHSDDTDDLSYLTALLRGYLIGLDHIPHSAIGLEDNASASPLLGIRSWQTRALLIKALIDQGYMKQILVSNDWLFGFSSYVTNIMDVMDRVNPDGMAFIH

SEQ ID NO: 2 - protein, 4 aa, Artificial Sequence
  Synthetic peptide
SIGT

SEQ ID NO: 3 - protein, 335 aa, Unknown
  Organophosphorus hydrolase of unknown origin
MIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDRVNPDGMAFIPLRVIPFLREKGVPQETLAGITVTNPARFLSPTLRA

SEQ ID NO: 4 - protein, 336 aa, Unknown
  Organophosphorus hydrolase of unknown origin
MIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIKDTGIRAGIIKVATTGKATPFQELVLKAAARASLATGVPVTTHTAASQRDGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDRVNPDGMAFIPLRVIPFLREKGVPQETLAGITVTNPARFLSPTLRAS

(19) United States
(12) Patent Application Publication
(10) Pub. No.: US 2011/0262996 A1
(43) Pub. Date: Oct. 27, 2011

(54) METHODS AND MATERIALS FOR DEGRADING PYRETHROID

(75) Inventor: Carmen Costable, Marion, IA (US)

(73) Assignees: DANISCO A/S, Copenhagen K (DK); DANISCO US INC., Rochester, NY (US)

(21) Appl. No.: 13/055,127

(22) PCT Filed: Apr. 29, 2009
(86) PCT No.: PCT/EP2009/055226

§ 371 Date: Jul. 13, 2011

Related U.S. Application Data
(60) Provisional application No. 61/125,967, filed on Apr. 30, 2008.

(51) Int. Cl. A62D 3/02; C12N 9/16

(52) U.S. Cl. 435/196; 435/262.5

(57) ABSTRACT

Methods and compositions are provided for enzymatic detoxification of pyrethroid such as permethrin. The methods involve application of compositions of organophosphorus hydrolase OPH to surfaces or substances contaminated with pyrethroid such as permethrin.

### REFERENCE TO A SEQUENCE LISTING

A sequence listing, comprising SEQ ID NOS: 1-4, is attached and is incorporated by reference in its entirety.

### FIELD OF THE INVENTION

Methods for degrading pyrethroid such as permethrin, and enzymatic compositions for conducting the degradation, among other things.

### BACKGROUND

Pyrethroids are a group of man-made pesticides similar to the natural pesticide pyrethrum, which is produced by chrysanthemum flowers. Pyrethroids are widely used for controlling various insects. Pyrethroids inhibit the nervous system of insects. There are two types that differ in chemical structure and symptoms of exposure. Type I pyrethroids include allethrin, tetramethrin, resmethrin, d-phenothrin, bioresmethrin, and permethrin. Some examples of type II pyrethroids are cypermethrin, cyfluthrin, deltamethrin, cyphenothrin, fenvalerate, and fluvalinate. Both type I and II pyrethroids inhibit the nervous system of insects. This occurs at the sodium ion channels in the nerve cell membrane. Some pyrethroids also affect the action of a neurotransmitter called GABA. Pyrethroids are found in many commercial products used to control insects, including household insecticides, pet sprays and shampoos. Some pyrethroids also are used as lice treatments applied directly to the head and as mosquito repellents that can be applied to clothes.

Permethrin is a member of the pyrethroid class of pesticides. It is a United States Environmental Protection Agency (EPA) restricted-use pesticide for crop and wide area applications due to high toxicity to aquatic organisms, except for use as wide area mosquito adulticide. However, it is classified as a general use pesticide for residential and industrial applications. According to EPA data, approximately 2 million pounds of permethrin are applied annually to agricultural, residential, and public health uses sites.

Permethrin is known for its insect/arthropod repellent qualities, in addition to its relative safety when used by humans. Accordingly, another major use for Permethrin is as a moth-proofing agent in the manufacture of carpeting. As part of the manufacturing process, a permethrin-containing effluent is created. The resulting effluent creates handling and disposal problems for the carpet-production industry, because the effluent cannot merely be discarded into the environment, due to its potential toxicity.

Thus, caution must be exercised because permethrin is known to poison fish if it is introduced inadvertently into a stream or water cycle. In mammals, permethrin alters nerve function by modifying the normal biochemistry and physiology of nerve membrane sodium channels. Poisoning symptoms in humans include the irritation of skin and eyes, irritability to sound or touch, abnormal facial sensation, sensation of prickling, tingling or creeping on skin, numbness, headache, dizziness, nausea, vomiting, diarrhea, excessive salivation, and fatigue.

There is therefore a need for a pyrethroid decontaminant such as a permethrin decontaminant that is non-toxic, environmentally-safe and user friendly, capable of detoxifying permethrin in effluent, as well as in other solutions and mixtures. A pyrethroid decontaminant such as a permethrin decontaminant that can act on permethrin that is not in solution is also needed. The disclosure set forth herein meets and addresses these needs.

### SUMMARY

Provided herein is a method of degrading pyrethroid such as permethrin, the method comprising contacting pyrethroid with a composition comprising the enzyme organophosphate hydrolase (OPH). There is further herein provided a method of degrading pyrethroid such as permethrin in a sample, the method comprising contacting the sample with a composition comprising the enzyme organophosphate hydrolase (OPH). In an aspect, the pyrethroid such as permethrin is completely degraded. In another aspect, the pyrethroid such as permethrin is partially degraded.

Also provided herein is a method of inactivating pyrethroid such as permethrin in a sample, the method comprising contacting the sample with a composition comprising OPH.

Also provided herein is a method of rendering pyrethroid such as permethrin less active in a sample, the method comprising contacting the sample with a composition comprising OPH. In an aspect, the sample has a pH in the range of about 6.5 to about 9.0. In another aspect, the pH is about 8.5. In another aspect, the pH of the sample is adjusted to 8.5. In an aspect, the temperature of the sample is in the range of about 20° C.-30° C. In another aspect, the temperature of the sample is about 22° C.

In an aspect, a pyrethroid-containing sample such as a permethrin-containing sample is effluent from a manufacturing process. In an aspect, the manufacturing process comprises the treatment of carpeting with pyrethroid such as permethrin.

Also provided herein is a method of degrading pyrethroid such as permethrin in a sample, the method comprising contacting the sample with a composition comprising a microbe that expresses OPH. In an aspect, the microbe is a bacterium.

Also provided herein is a pyrethroid degrading composition such as a permethrin-degrading composition, the composition comprising at least one isolated enzyme. In an aspect, a pyrethroid-degrading composition such as a permethrin-degrading composition comprises OPH. In an aspect, use of OPH for degrading pyrethroid is provided.

### BRIEF DESCRIPTION OF THE DRAWINGS

The accompanying drawings are incorporated in and constitute a part of this specification and illustrate various embodiments.

FIG. 1 is a graphical depiction of the amount of permethrin remaining in a sample over a fifteen minute time course.

FIG. 2 illustrates the activity of OPH as a function of temperature, for permethrin degradation by OPH. OPH activity is expressed as OPH units per gram of enzyme.

FIG. 3 illustrates the activity of OPH as a function of pH, for permethrin degradation by OPH. OPH activity is expressed as OPH units per gram of enzyme.

FIG. 4 illustrates the activity of OPH as a function of buffer concentration, for permethrin degradation by OPH. OPH activity is expressed as percent relative activity.

### DETAILED DESCRIPTION

Provided herein is a method of degrading pyrethroid, the method comprising contacting pyrethroid with a composition comprising the enzyme organophosphate hydrolase (OPH).

Furthermore a method of degrading pyrethroid such as permethrin, wherein the pyrethroid is present in a sample, the method comprising contacting the sample with a composition comprising the enzyme organophosphate hydrolase (OPH) is provided.

In one aspect, the pyrethroid is completely degraded or at least partially degraded.

Pyrethroids are known for possessing insecticidal properties (see e.g. US 2007/0276013 A1, WO 2005077186, WO 93-22 297, WO 93-10 083, DE-A 2 641 343, EP-A-347 488, EP-A-210 487, U.S. Pat. No. 3,264,177 and EP-A-234 045).

In one aspect, the pyrethroid is selected from the group consisting of acrinathrin known from EP-A-048 186, alpha-cypermethrin known from EP-A-067 46, betacyfluthrin known from EP-A-206 149, cyhalothrin known from DE-A-2 802 962, cypermethrin known from DE-A-2 326 077, deltamethrin known from DE-A-2 326 077, esfenvalerate known from DE-A-2 737 297, ethofenprox known from DE-A-3 117 510, fenpropathrin known from DE-A-2 231 312, fenvalerate known from DE-A-2 335 347, flucythrinate known from DE-A-2 757 066, lambda-cyhalothrin known from EP-A-106 469, permethrin known from DE-A-2 326 077, taufluvalinate known from EP-A-038 617, tralomethrin known from DE-A-2 742 546, zeta-Cypermethrin known from EP-A-026 542, cyfluthrin known from DE-A-27 09 264, bifenthrin known from EP-A-049 977, cycloprothrin known from DE-A-2653189, eflusilanate known from DE-A-36 04 781, fubfenprox known from DE-A-37 08 231, pyrethrin known from The Pesticide Manual 1997, issue 11, page 1056, resmethrin known from GB-A-1 168 797, gamma-cyhalothrin (known from “The Pesticide Manual, 13.th Edition, 2003, entry 197 on page 232, by The British Crop Protection Council) and sumithrin.

In one aspect, the pyrethroid is selected from the group consisting of acrinathrin, allethrin (d-cis-trans, d-trans), beta-cyfluthrin, bifenthrin, bioallethrin, bioallethrin-5-cyclopentyl-isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, taufluvalinate, tefluthrin, terallethrin, tetramethrin (−1R-isomer), tralomethrin, transfluthrin, ZXI 8901, pyrethrins (pyrethrum).

In one aspect, the pyrethroid is selected from the group consisting of acrinathrin, alpha-cypermethrin, betacyfluthrin, cyhalothrin, cypermethrin, deltamethrin, esfenvalerate, ethofenprox, fenpropathrin, fenvalerate, flucythrinate, lambda-cyhalothrin, permethrin, taufluvalinate, fluvalinate, tralomethrin, zeta-cypermethrin, cyfluthrin, bifenthrin, cycloprothrin, eflusilanate, fubfenprox, pyrethrin, resmethrin, gamma-cyhalothrin, allethrin, tetramethrin, d-phenothrin, bioresmethrin, cyphenothrin and sumithrin.

In one aspect, the pyrethroid is selected from the group consisting of allethrin, tetramethrin, resmethrin, d-phenothrin, bioresmethrin, and permethrin.

In one aspect, the pyrethroid is selected from the group consisting of cypermethrin, cyfluthrin, deltamethrin, cyphenothrin, fenvalerate, and fluvalinate.

In one aspect, the pyrethroid is selected from the group consisting of permethrin, resmethrin, and sumithrin.

In one aspect, the pyrethroid is permethrin.

Permethrin is an example of a pyrethroid which is a widely-used compound, such as in agriculture and in material production and processing, among other things. However, permethrin as well as other pyrethroids is toxic to fish and various mammals, and therefore, care must be taken with the handling and disposal of permethrin-containing waste from industrial and commercial uses of permethrin. For example, permethrin-containing effluent from the carpet manufacturing process presents a handling and disposal problem. It is shown herein that the enzyme organophosphate hydrolase (OPH) can degrade permethrin. By using a catalytically-efficient enzyme to process permethrin waste and permethrin-containing compositions, the use of additional toxic or costly chemicals can be avoided.

### 1. Definitions and Abbreviations

In accordance with this detailed description, the following abbreviations and definitions apply. It should be noted that as used herein, the singular forms “a,” “an,” and “the” include plural referents unless the context clearly dictates otherwise. Thus, for example, reference to “an enzyme” includes a plurality of such enzymes, and reference to “the formulation” includes reference to one or more formulations and equivalents thereof known to those skilled in the art, and so forth.

Unless defined otherwise, all technical and scientific terms used herein have the same meaning as commonly understood by one of ordinary skill in the art. The following terms are provided below.

### 1.1. Definitions

As used herein, the term “expression” refers to the process by which a polypeptide is produced based on the nucleic acid sequence of a gene. The process includes both transcription and translation.

“Isolated” means that the sequence is at least substantially free from at least one other component that the sequence is naturally associated and found in nature, e.g., genomic sequences.

“Purified” means that the material is in a relatively pure state, e.g., at least about 90% pure, at least about 95% pure, or at least about 96%, 97%, 98%, or 99% pure.

As used herein, “amino acid sequence” is synonymous with the term “polypeptide” and/or the term “protein.” In some instances, the term “amino acid sequence” is synonymous with the term “peptide”; in some instances, the term “amino acid sequence” is synonymous with the term “enzyme.”

As used herein, “nucleotide sequence” or “nucleic acid sequence” refers to an oligonucleotide sequence or polynucleotide sequence and variants, homologues, fragments and derivatives thereof. The nucleotide sequence may be of genomic, synthetic or recombinant origin and may be double-stranded or single-stranded, whether representing the sense or anti-sense strand. As used herein, the term “nucleotide sequence” includes genomic DNA, cDNA, synthetic DNA, and RNA.

As used herein, a “synthetic” compound is produced by in vitro chemical or enzymatic synthesis. It includes, but is not limited to, variant nucleic acids made with optimal codon usage for host organisms, such as a yeast cell host or other expression hosts of choice. As used herein, “OPH activity” refers to the degradation of permethrin as set forth herein.

A “sample”, as the term is used herein, is any substance that can contain permethrin.

### 1.2. Abbreviations

The following abbreviations apply unless indicated otherwise:

cDNA complementary DNA
DEAE diethylamino ethanol
DNA deoxyribonucleic acid
EC enzyme commission for enzyme classification
GCMS gas chromatography mass spectrometry
HPLC high performance liquid chromatography
mRNA messenger ribonucleic acid
OP organophosphate
OPH organophosphate hydrolase
PCR polymerase chain reaction
ppm parts per million
RT-PCR reverse transcriptase polymerase chain reaction
SDS-PAGE sodium dodecyl sulfate-polyacrylamide gel electrophoresis
1×SSC 0.15 M NaCl, 0.015 M sodium citrate, pH 7.0
w/v weight/volume
w/w weight/weight

### 2. Methods and Compositions for Decontamination of Pyrethroid

2.1. Compositions

Compositions disclosed herein for decontamination of pyrethroid such as permethrin comprise the enzyme organophosphate hydrolase (OPH). OPH, also known as phosphotriesterase, is shown herein to degrade permethrin. An enzymatic method of decontamination, as disclosed herein, offers considerable advantages over other decontaminants. Being catalytic, an enzyme is highly efficient and can detoxify many times its own weight of contaminating agent in minutes or even seconds.

OPH, originally referred to as parathion hydrolase, is an enzyme found in a number of bacterial isolates. OPH has activity against a variety of organophosphate (OP) compounds, including chemical warfare nerve agents. Many investigators have studied OPH, and information related to the structure and function can be found in the art (e.g., Mulbry et al., J. Bacteriol. (1989) 171: 6740-6746; Raushel, Curr. Opin. Microbiol. (2002) 5: 288-295).

In one aspect, “organophosphate hydrolase”, “OPH”, “phosphotriesterase”, “PTE”, as used herein refers to any aryldialkylphosphatase (E.C. 3.1.8.1), which has the ability to act on organophosphorus compounds (such as paraoxon) including esters of phosphonic and phosphinic acids, such as in the hydrolysis of an organophosphate (OP).

Other names used commonly for OPH include: paraoxonase; A-esterase; aryltriphosphatase; organophosphate esterase; esterase B1; esterase E4; paraoxon esterase; pirimiphos-methyloxon esterase; OPA anhydrase; organophosphorus hydrolase; paraoxon hydrolase; organophosphorus acid anhydrase.

The gene for this enzyme has been cloned, sequenced, and expressed in a number of prokaryotic and eukaryotic host organisms. Two common sources for OPH enzyme are the identical opd genes isolated from Pseudomonas diminuta MG and the Flavobacterium sp. strain ATCC 27551. The P. diminuta MG opd gene was isolated by McDaniel et al. ((1989) J. Bacteriol., 170: 2306-2311; incorporated by reference herein in its entirety). The McDaniel et al. opd gene is referenced in Genebank, with ascension number M20392, and incorporated by reference herein in its entirety, as follows: LOCUS PSEPTE 1322 bp DNA BCT Apr. 21, 1996 DEFINITION Plasmid pCMS1 (from P. diminuta) phosphodiesterase (opd) gene, complete cds. ACCESSION M20392 NID g151517 VERSION M20392.1 GI:151517. The amino acid sequence for the P. diminuta sequence is set forth in SEQ ID NO:1.

The open reading frame of the opd gene, as reported by McDaniel et al., contains 975 bases which encode an OPH polypeptide of 325 amino acid residues with a molecular mass of 35 kDa. Mulbry et al. (J. Bacteriol. (1989) 171: 6740-6746; incorporated by reference herein in its entirety) also cloned the opd gene, but that clone lacked 4 amino-terminal residues (Ser-Ile-Gly-Thr or SIGT) (SEQ ID NO: 2), relative to the opd gene described above.

The amino acid sequence for the organophosphorus hydrolase Flavobacterium sp. MTCC 2495 is set forth in SEQ ID NO:4. The amino acid sequence set forth in SEQ ID NO:3 differs 1 amino acid from SEQ ID NO:4 and is lacking a serine at the end.

In a further aspect, the OPH comprises the amino acid sequence having SEQ ID No. 1. In a further aspect, the OPH comprises the amino acid sequence having SEQ ID No. 3. In a further aspect, the OPH comprises the amino acid sequence having SEQ ID No. 4.

The three-dimensional crystal structure of OPH also has been determined revealing that the native enzyme is a homodimer containing two Zn²⁺ ions per subunit. The Co²⁺ substituted enzyme has greater activity on nerve agents and substrates with P—F and P—S bonds (Omburo et al., J. Biol. Chem. (1992) 267: 13278-13283). While more research has been conducted on OPH compared to other chemical agent-degrading enzymes, its cellular function and native substrate remain unknown.

In one aspect, the use of polypeptides having a degree of sequence identity or sequence homology with amino acid sequence(s) defined herein or with a polypeptide having the specific properties defined herein is provided. In one aspect, peptides having a degree of sequence identity with SEQ ID No. 1, SEQ ID No. 3 or SEQ ID No. 4 or homologues thereof are provided. Here, the term “homologue” means an entity having sequence identity with the subject amino acid sequences or the subject nucleotide sequences. Here, the term “homology” can be equated with “sequence identity”.

The homologous amino acid sequence and/or nucleotide sequence should provide and/or encode a polypeptide which retains the functional activity and/or enhances the activity of the OPH enzyme.

In the present context, a homologous sequence is taken to include an amino acid sequence which may be at least 50%, preferably at least 55%, such as at least 60%, for example at least 65%, at least 70%, at least 75%, at least 80%, at least 85%, at least 90%, at least 95%, at least 96%, at least 97%, at least 98% or at least 99%, identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In one embodiment, the OPH enzyme is an organophosphorus hydrolase having the sequence shown in SEQ ID No 1, 3 or 4 or a sequence having at least 50%, preferably at least 55%, such as at least 60%, for example at least 65%, at least 70%, at least 75%, at least 80%, at least 85%, at least 90%, at least 95%, at least 96%, at least 97%, at least 98% or at least 99%, sequence identity therewith.

Sequence identity comparisons can be conducted by eye, or more usually, with the aid of readily available sequence comparison programs. These commercially available computer programs use complex comparison algorithms to align two or more sequences that best reflect the evolutionary events that might have led to the difference(s) between the two or more sequences. Therefore, these algorithms operate with a scoring system rewarding alignment of identical or similar amino acids and penalising the insertion of gaps, gap extensions and alignment of non-similar amino acids. The scoring system of the comparison algorithms include:
i) assignment of a penalty score each time a gap is inserted (gap penalty score),
ii) assignment of a penalty score each time an existing gap is extended with an extra position (extension penalty score),
iii) assignment of high scores upon alignment of identical amino acids, and
iv) assignment of variable scores upon alignment of non-identical amino acids.

Most alignment programs allow the gap penalties to be modified. However, it is preferred to use the default values when using such software for sequence comparisons.

The scores given for alignment of non-identical amino acids are assigned according to a scoring matrix also called a substitution matrix. The scores provided in such substitution matrices are reflecting the fact that the likelihood of one amino acid being substituted with another during evolution varies and depends on the physical/chemical nature of the amino acid to be substituted. For example, the likelihood of a polar amino acid being substituted with another polar amino acid is higher compared to being substituted with a hydrophobic amino acid. Therefore, the scoring matrix will assign the highest score for identical amino acids, lower score for non-identical but similar amino acids and even lower score for non-identical non-similar amino acids. The most frequently used scoring matrices are the PAM matrices (Dayhoff et al. (1978), Jones et al. (1992)), the BLOSUM matrices (Henikoff and Henikoff (1992)) and the Gonnet matrix (Gonnet et al. (1992)).

Suitable computer programs for carrying out such an alignment include, but are not limited to, Vector NTI (Invitrogen Corp.) and the ClustalV, ClustalW and ClustalW2 programs (Higgins D G & Sharp P M (1988), Higgins et al. (1992), Thompson et al. (1994), Larkin et al. (2007). A selection of different alignment tools are available from the ExPASy Proteomics server at www.expasy.org. Another example of software that can perform sequence alignment is BLAST (Basic Local Alignment Search Tool), which is available from the webpage of National Center for Biotechnology Information which can currently be found at http://www.ncbi.nlm.nih.gov/ and which was firstly described in Altschul et al. (1990) J. Mol. Biol. 215; 403-410.

Once the software has produced an alignment, it is possible to calculate % similarity and % sequence identity. The software typically does this as part of the sequence comparison and generates a numerical result.

In one embodiment, it is preferred to use the ClustalW software for performing sequence alignments. Preferably, alignment with ClustalW is performed with the following parameters for pairwise alignment:

| | |
|---|---|
| Substitution matrix: | Gonnet 250 |
| Gap open penalty: | 20 |
| Gap extension penalty: | 0.2 |
| Gap end penalty: | None |

ClustalW2 is for example made available on the internet by the European Bioinformatics Institute at the EMBL-EBI webpage www.ebi.ac.uk under tools-sequence analysis-ClustalW2. Currently, the exact address of the ClustalW2 tool is www.ebi.ac.uk/Tools/clustalw2.

Thus, provided herein is the use of variants, homologues and derivatives of any amino acid sequence of a protein as defined herein, particularly those of SEQ ID No. 1, 3 or 4.

The sequences, particularly those of SEQ ID No. 1, 3 or 4, may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Also provided herein is a conservative substitution (substitution and replacement are both used herein to mean the interchange of an existing amino acid residue, with an alternative residue) that may occur i.e. like-for-like substitution such as basic for basic, acidic for acidic, polar for polar etc. Non-conservative substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Conservative substitutions that may be made are, for example within the groups of basic amino acids (Arginine, Lysine and Histidine), acidic amino acids (glutamic acid and aspartic acid), aliphatic amino acids (Alanine, Valine, Leucine, Isoleucine), polar amino acids (Glutamine, Asparagine, Serine, Threonine), aromatic amino acids (Phenylalanine, Tryptophan and Tyrosine), hydroxyl amino acids (Serine, Threonine), large amino acids (Phenylalanine and Tryptophan) and small amino acids (Glycine, Alanine).

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#*}, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-conservative substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al. (1992), Horwell D C. (1995).

In one embodiment, the OPH enzyme is an organophosphorus hydrolase having the amino acid sequence shown in SEQ ID No 1, 3 or 4 or an enzyme having at least 50%, preferably at least 55%, such as at least 60%, for example at least 65%, at least 70%, at least 75%, at least 80%, at least 85%, at least 90%, at least 95%, at least 96%, at least 97%, at least 98% or at least 99% amino acid sequence identity therewith.

In one aspect, the sequence used in the present invention is in a purified form. The component is desirably the predominant active component present in a composition.

Recombinant OPH enzyme can be purified and isolated using any means known in the art. In one embodiment, OPH is prepared and purified through cloning in an expression vector within Escherichia coli host, as set forth in U.S. Pat. No. 6,469,145, incorporated herein by reference in its entirety. Briefly, OPH is expressed from a suitable cell-based expression system. In one embodiment, OPH is expressed from a bacterial cell expression system. The aqueous solution of bacterial proteins is contacted with a strong cation exchange resin. The strong cation exchange resin is then washed with a washing buffer to remove unbound proteins, and the OPH specifically eluted using an eluting buffer of increasing salt concentration. The resulting eluate can be assayed, confirmed to contain OPH, then stored using methods known in the art.

Compositions of OPH useful as set forth herein can be prepared as described in the experimental examples. Compositions comprising OPH may include one or more of buffers, salts, stabilizers, preservatives, and other components as necessary, and as will be appreciated by the skilled artisan, in order to maximize OPH activity. “OPH” activity refers to the degradation of permethrin as set forth herein.

In one embodiment, a composition comprising OPH is a microbial culture or a microbe-containing medium, wherein the microbe expresses OPH. In one aspect, the microbe expresses OPH and secretes the OPH into the surrounding medium. In another aspect, the microbial membrane is disrupted or otherwise compromised in order to release the expressed OPH into solution. In one embodiment, the microbe is a bacterium, such as, but not limited to, Escherichia coli.

It will be understood that when an OPH decontaminating composition set forth herein is contacted with a sample in which e.g. permethrin is to be contaminated, the physical and/or chemical properties of the sample may be adjusted as necessary to optimize the activity of the OPH. The experimental examples set forth herein provide guidance to the skilled artisan as to the time, temperature, concentration, and pH values which provide improved or optimal OPH activity with respect to the degradation of pyrethroid such as permethrin.

In one aspect, the sample has a pH in the range of about 6.5 to about 9.0. In a further aspect, the pH is about 8.5. In one aspect, the temperature of the sample is in the range of about 20° C.-30° C. In a further aspect, the temperature of the sample is about 22° C.

In one aspect, the composition comprising OPH include a buffer. In a further aspect, the buffer concentration is at least 10 mM buffer. In a further aspect, the buffer concentration is at least 50 mM buffer. In a further aspect, the buffer concentration is at least 90 mM buffer. In one aspect, the buffer is ammonium carbonate buffer.

An additional advantage of the disclosure set forth herein is that an OPH enzyme-based decontaminant will pose little or no health or environmental danger and will result in no hazardous products requiring cleanup. In an aspect, an OPH-comprising composition as set forth herein is non-toxic and not harmful to living organisms.

Another major advantage is that an OPH enzyme-based decontaminant can be provided as a dry powder that can be added to any available water-based spray or foam systems available to the user. This provides a significant reduction in the logistical burden for use of the permethrin-decontaminating composition.

2.2. Methods

In one embodiment, OPH degrades pyrethroid such as permethrin. Provided herein is a method of degrading pyrethroid such as permethrin in a sample. A “sample”, as the term is used herein, is any substance containing pyrethroid such as permethrin. A sample may contain pyrethroid such as permethrin on the outer surface of the sample, on the inside of the sample, or integrated into the sample. Samples include, but are not limited to, a solid sample (e.g., soil, gravel, wood, clothing, carpeting, etc. . . . ), a semi-solid sample, and a liquid sample (e.g., water from a lake, stream, or river, effluent from a manufacturing process, sewage run-off, etc. . . . ), a living organism among others.

A sample may be identified as containing e.g. permethrin by testing the sample for the presence of permethrin. As set forth elsewhere herein, permethrin can e.g. be detected using mass spectrometry and/or gas chromatography. However, the skilled artisan will understand that many methods of detecting pyrethroid such as permethrin are available, and all such methods, either now known or yet to be discovered, are encompassed herein.

Similarly, the extent of degradation or removal of pyrethroid such as permethrin in a sample may be monitored after treatment according to the disclosure herein by testing the sample for the presence of permethrin, and comparing the concentration of permethrin to the concentration of permethrin in the same sample prior to treatment according to the methods herein.

Therefore, provided herein are methods for detoxifying, decontaminating, altering, removing or reducing the concentration of pyrethroid such as permethrin in an aqueous solution, leachate, runoff, aquifer, groundwater, surface water, well water, an environmental site, soils, agricultural or industrial samples, and/or sites including industrial holding ponds, waste water treatment facilities, and water sources in mosquito abatement areas.

In one embodiment, a pyrethroid-containing sample such as a permethrin-containing sample of effluent from a manufacturing process is contacted with an OPH-containing composition to degrade the permethrin. In an exemplary embodiment, a pyrethroid-containing sample such as a permethrin-containing sample of effluent from a carpet manufacturing and treatment process is contacted with an OPH-containing composition to degrade the pyrethroid e.g. the permethrin.

A method of treating a liquid sample comprises, in one aspect, the addition of an OPH-comprising composition to the liquid sample. The composition added may be a solid, a liquid, or microbial, or a combination thereof. In an aspect, the OPH-comprising composition is non-toxic. In one embodiment, because an OPH-comprising composition may be non-toxic, the composition need not be recovered from the sample, nor remediated further. In an aspect, a method of treating a liquid sample comprises treating a static liquid sample. In another aspect, a method of treating a liquid sample comprises treatment of a continuous-flow sample by contacting the sample with an OPH-comprising composition.

A method of treating a solid sample comprises, in one aspect, the addition of an OPH-comprising composition the solid sample. In an aspect, the OPH-comprising composition is non-toxic. In one embodiment, because an OPH-comprising composition may be non-toxic, the composition need not be recovered from the solid sample, nor the solid sample remediated further. In an aspect, a method of treating a solid sample comprises addition an OPH-comprising composition to the solid sample. The composition may be a solid, a liquid, or microbial, or a combination thereof.

As set forth elsewhere herein, a method of degrading pyrethroid such as permethrin in a sample comprises contacting the sample with a composition comprising the enzyme OPH. Therefore, in one aspect, the pyrethroid such as the permethrin in a sample is completely degraded by OPH. In another aspect, the pyrethroid such as the permethrin in a sample is partially degraded by OPH.

Also disclosed herein is a method of inactivating pyrethroid such as permethrin, comprising contacting the pyrethroid with a composition comprising OPH. A method of rendering pyrethroid less active is provided, comprising contacting the pyrethroid with a composition comprising OPH. Also disclosed herein is a method of inactivating pyrethroid such as permethrin in a sample, comprising contacting the sample with a composition comprising OPH. A method of rendering pyrethroid such as permethrin less active in a sample is provided, comprising contacting the sample with a composition comprising OPH.

In any method of degrading or inactivating pyrethroid such as permethrin embodied herein, a sample has a pH in the range of 6.5-10.0. In another embodiment, a sample has a pH in the range of 8.0-9.0. In an exemplary embodiment, the pH is 8.5. The pH of the sample may be suitable for sufficient or optimal enzyme activity. In one aspect, the pH of the sample is adjusted to 8.5.

In any method of degrading or inactivating pyrethroid such as permethrin embodied herein, a sample has a temperature suitable for sufficient or optimal enzyme activity. However, it will be understood that the temperature of the sample may be adjusted to optimize OPH activity. In an aspect, a temperature suitable for pyrethroid such as permethrin degradation by OPH is selected from the range of 2° C.-97° C. Exemplary temperature ranges include the range of 20° C.-30° C. In another embodiment, the temperature of the sample is 22° C.

In one embodiment, a method comprises contacting a sample with a composition comprising OPH, wherein the composition comprises microbe expressing OPH. In one aspect, a method comprises degrading pyrethroid such as permethrin by secretion the expressed OPH from the microbe into the sample. In another aspect, the method comprises degrading pyrethroid such as permethrin by disrupting, or otherwise compromising, the microbial membrane in order to release the expressed OPH into solution to degrade pyrethroid e.g. permethrin. In one embodiment of the method, the microbe is a bacterium, such as, but not limited to, Escherichia coli.

### EXPERIMENTAL EXAMPLES

### Example 1

### Degradation of Permethrin by OPH

OPH was assayed for the ability to degrade permethrin. Its efficacy has been measured by performing time course experiments wherein a sample of OPH is added to a sample of permethrin. Degraded permethrin is then separated from the reaction mixture and detected on a GCMS.

Experimental Methods and Equipment. A Shimadzu GC2010 Gas Chromatograph with AOC 20i Auto Injector and a Shimadzu GCMS QP2010S Quadrapole Mass Spectrometer were used to analyze the experimental samples. A Supelco Equity-5 column was used (length: 30.0 m, thickness: 0.25 μm, diameter: 0.25 mm) for the assays. However, analogous columns may be substituted.

The equipment was run with the following parameters:
column oven temperature: 120° C.
injector temperature: 250° C.
injection: splitless
pre-run rinses: 3
post-run rinses: 3
sample rinses: 2
linear velocity: 30 cm/sec
column flow: 0.64 mL/min
split ratio: 10
detector voltage: relative to tuning
ion source temperature: 250° C.
interface temperature: 270° C.
solvent cut time: 3 min
run time: 35 min
temperature ramp: 5° C./min
scan speed: 833

Experimental assays were run using recombinant OPH (DEFENZ™ 130G, lot 102-6181-001; Genencor). Reagents included ammonium carbonate (Sigma 207861, lot 12919JC), cobalt chloride (JT Baker 1670-04, lot Y41640), hydrochloric acid (JT Baker 5620-02, lot A23506), propanol (Sigma 154970, lot 04242AD), and permethrin (Sigma-Pestanal 45614, lot 7079×).

Preparation of Enzyme Samples. OPH enzyme granules (2% protein) were dissolved in 33 mM ammonium carbonate buffer, pH 8.5, at 22° C. The reaction mixture was agitated for 30 minutes, first vortexed, then slowly agitated on a temperature-controlled platform shaker. A 1.5 mL sample of the enzyme solution was taken and centrifuged for 1 minute at 13,000 rpm. An aliquot of the resulting supernatant is used in the pesticide degradation reaction.

Preparation of Permethrin Samples. A stock permethrin sample at a concentration of 100 mg/mL was made by dissolving an appropriate amount of permethrin standard into propanol. The stock sample was stored under refrigeration. For generating standard curves on the GCMS, permethrin standards were made by diluting an aliquot of the stock permethrin sample into propanol to achieve a concentration of 1 mg/mL and 10 mg/mL. Permethrin to be used in the enzyme reaction was prepared by diluting an aliquot of the stock solution into 33 mM ammonium carbonate buffer at the desired pH for a particular reaction, and at room temperature.

Enzyme Reactions. In a 1.5 mL tube, 250 μL of a 10 mg/mL permethrin solution in propanol was added to 0.75 mL of 33 mM ammonium carbonate buffer, pH 8.5. A 995 μL aliquot of the permethrin solution was placed in a 1.5 mL gas chromatography (GC) sample vial, to which was added a 5 μL aliquot of a 0.31 mg/mL OPH enzyme solution. To inactivate the reaction at any specific time point, a 10 μL aliquot of the reaction solution was removed and added to 990 μL of propanol in a GC sample vial. These samples were then loaded on the GCMS autosampler.

Results and Discussion. This study was conducted to determine whether OPH degrades permethrin. To quantitatively measure amounts of permethrin, the Shimadzu GC with Quadrapole MS detector was used, as in previous pesticide experiments. A standard curve was generated using 1 mg/mL and 10 mg/mL stock solutions in propanol. Table 1 describes the dosing scheme used for the curve. Regression of the standard curve yielded R2 values of 0.9997 above 50 ppm and of 0.998 below 50 ppm.

TABLE 1
Dosing of permethrin for generation of a standard curve.

| Curve | | 1 mg/ml permethrin | |
|---|---|---|---|
| point | ppm | (ml) | propanol (ml) |
| 0 | 0 | 0.000 | 1.000 |
| 1 | 1 | 0.001 | 0.999 |
| 2 | 2 | 0.002 | 0.998 |
| 3 | 5 | 0.005 | 0.995 |
| 4 | 7 | 0.007 | 0.993 |
| 5 | 15 | 0.015 | 0.985 |
| 6 | 25 | 0.025 | 0.975 |

| | | 10 mg/ml permethrin | |
|---|---|---|---|
| — | ppm | (ml) | propanol (ml) |
| 7 | 10 | 0.001 | 0.999 |
| 8 | 50 | 0.005 | 0.995 |
| 9 | 100 | 0.010 | 0.990 |
| 10 | 250 | 0.025 | 0.975 |
| 11 | 500 | 0.050 | 0.950 |

Preliminary experiments were conducted to ascertain optimum concentrations for the enzyme reactions. It was found that a stock enzyme concentration of 0.31 mg/mL, diluted further in the reaction in which 5 μL was added to 995 μL of a 2.5 mg/mL pesticide solution, resulted in measurable amounts of un-degraded pesticide. Samples were taken at time points of 0, 1, 5, 15, and 30 minutes by removing 10 μL of the reaction mixture and adding it to 990 μL of propanol. Table 2 describes the amount of pesticide quantified at each time point using the detector. It is noted that there is an initial, rapid degradation of approximately 6 ppm permethrin at the detector, occurring within 1 minute of the start of the reaction. Thereafter, the reaction rate proceeds to a degraded amount of permethrin of 12 ppm at the detector after 30 minutes. These results demonstrate that 1 g of DEFENZ 130G will degrade 790 g of permethrin in 30 minutes at pH 8.5 and room temperature (approximately 22° C.).

However, it will be understood that the enzyme concentration can be increased or decreased to the point at which the desired permethrin-degrading activity is obtained. In an aspect, the enzyme concentration may range from 0.1% w/v to 20% w/v.

TABLE 2
Amount of Permethrin measured at various time points.

| | Time (min.) | | | | |
|---|---|---|---|---|---|
| | 0 | 1 | 5 | 15 | 30 |
| ppm permethrin | 31.47 | 25.8 | 25.88 | 24.64 | 19.27 |

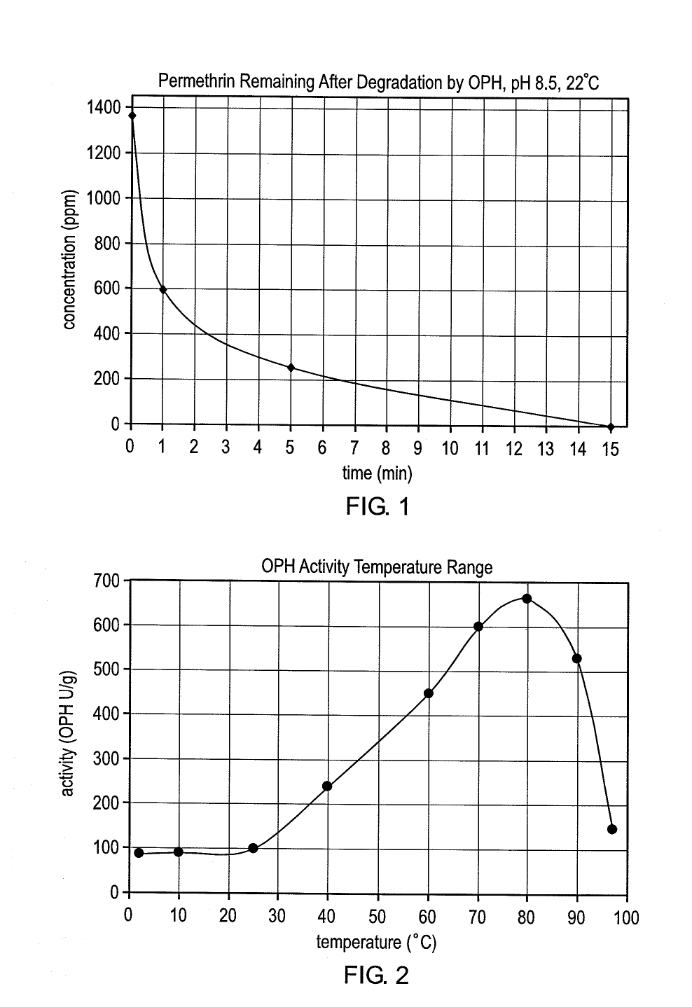

FIG. 1 is a graphical depiction of the amount of permethrin remaining in a sample over a fifteen minute time course. The assay was conducted using approximately one gram of OPH (DEFENZ130/PestDegrade; Genencor). As illustrated in the graph, approximately one gram of OPH will break down approximately 445 grams of permethrin in fifteen minutes at pH 8.5, 22° C. Based on the data presented herein, it will be understood that the amount of time the reaction will be allowed to proceed is based on the desired outcome (i.e., the amount of permethrin to be degraded), in view of the reaction parameters (e.g., temperature, pH, etc.).

FIG. 2 illustrates the activity of OPH as a function of temperature, for permethrin degradation by OPH. OPH activity is expressed as OPH units per gram of enzyme. The assay was conducted at pH 8.5. FIG. 2 illustrates that under the specific assay conditions, 80° C. provides the maximal permethrin-degrading activity. However, the enzyme still actively degrades permethrin at temperatures as low as 2° C.

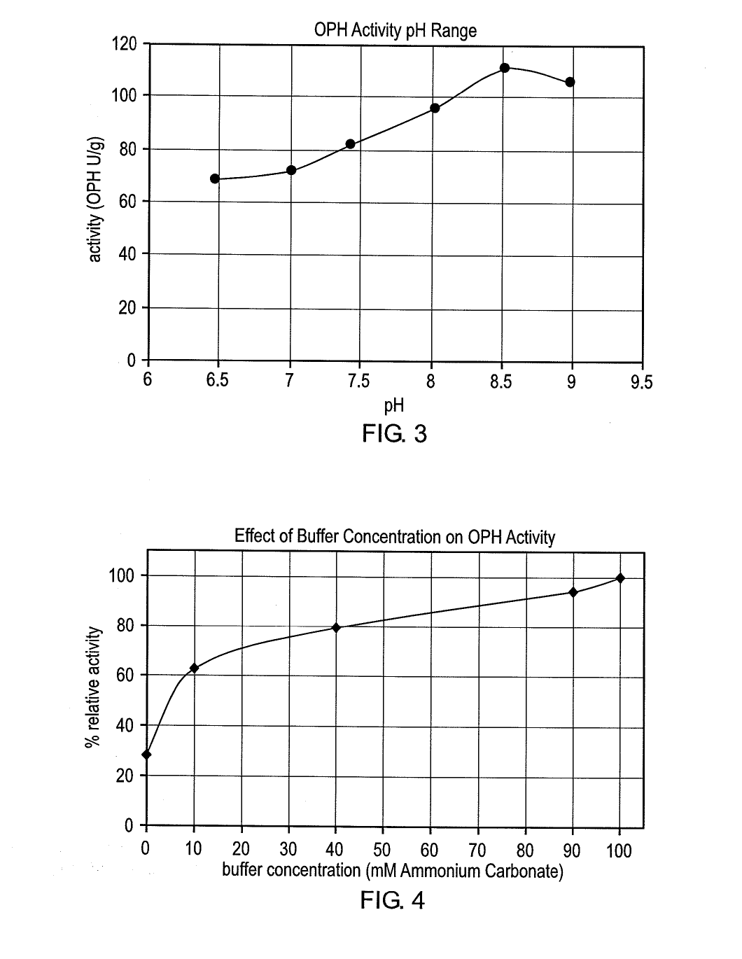

FIG. 3 illustrates the activity of OPH as a function of pH, for permethrin degradation by OPH. OPH activity is expressed as OPH units per gram of enzyme. The assay was conducted at 22° C. FIG. 3 illustrates that OPH actively degrades permethrin over the entire measured range from pH 6.5 to pH 9.0, showing maximal activity under these assay conditions at pH 8.5.

FIG. 4 illustrates the activity of OPH as a function of buffer concentration, for permethrin degradation by OPH. OPH activity is expressed as percent relative activity. It will be understood from the data presented herein that “100%” OPH activity can be assigned in one of many ways. For example, a determination of 100% activity can be based on the desired outcome (i.e., the amount of permethrin to be degraded), in view of the reaction parameters (e.g., temperature, pH, etc. . . . ). 100% activity is the highest activity measured for a given set of reaction conditions. In the experiments exemplified in FIG. 4, 100% activity occurs at a buffer concentration of 100 mM, though permethrin degrading activity is detected from 0 mM ammonium carbonate through 100 mM ammonium carbonate.

### SEQUENCES

| SEQ ID No. 1 | |
|---|---|
| MQTRRVVLKS AAARTLLGGL AGCATWLDRS AQAMRSIRAR PITISEAGFT LTHEDISAAR | 60 |
| QDSCVLGQSS SVAQSSSGKG CERIARQSGW RANDCRCVDF RYRSRRQFIG RGFAGCRRSY | 120 |
| LAATGLWFDP PLSMRLRYVE ELTLVLPAVR FNMASKYTGI RAGIIKVATT GKATPFQELV | 180 |
| LKAAARASLA TGVPVTTHTA ASQRDGERGR PPFLSPKLEP SRVCIGHSDD TDDLSYLTAL | 240 |
| LRGYLIGLDH IPHSAIGLED NASASPLLGI RSWQTRALLI KALIDQGYMK QILVSNDWLF | 300 |
| GFSSYVTNIM DVMDRVNPDG MAFIH | 325 |
| SEQ ID No. 2 | |
| SIGT | |
| SEQ ID No. 3 | |
| MIGTGDRINT VRGPITISEA GFTLTHEHIC GSSAGFLRAW PEFFGSRKAL AEKAVRGLRR | 60 |
| ARAAGVRTIV DVSTFDIGRD VSLLAEVSRA ADVHIVAATG LWFDPPLSMR LRSVEELTQF | 120 |
| FLREIQYGIE DTGIRAGIIK VATTGKATPF QELVLKAAAR ASLATGVPVT THTAASQRDG | 180 |
| EQQAAIFESE GLSPSRVCIG HSDDTDDLSY LTALAARGYL IGLDHIPHSA IGLEDNASAS | 240 |
| ALLGIRSWQT RALLIKALID QGYMKQILVS NDWLFGFSSY VTNIMDVMDR VNPDGMAFIP | 300 |
| LRVIPFLREK GVPQETLAGI TVTNPARFLS PTLRA | 335 |
| SEQ ID No. 4 | |
| MIGTGDRINT VRGPITISEA GFTLTHEHIC GSSAGFLRAW PEFFGSRKAL AEKAVRGLRR | 60 |
| ARAAGVRTIV DVSTFDIGRD VSLLAEVSRA ADVHIVAATG LWFDPPLSMR LRSVEELTQF | 120 |
| FLREIQYGIK DTGIRAGIIK VATTGKATPF QELVLKAAAR ASLATGVPVT THTAASQRDG | 180 |
| EQQAAIFESE GLSPSRVCIG HSDDTDDLSY LTALAARGYL IGLDHIPHSA IGLEDNASAS | 240 |
| ALLGIRSWQT RALLIKALID QGYMKQILVS NDWLFGFSSY VTNIMDVMDR VNPDGMAFIP | 300 |
| LRVIPFLREK GVPQETLAGI TVTNPARFLS PTLRAS | 336 |

All patents, patent applications, and published references cited herein are hereby incorporated by reference in their entirety. The disclosure set forth herein has been particularly shown and described with references to preferred embodiments thereof, it will be understood by those skilled in the art that various changes in form and details may be made therein without departing from the scope encompassed by the present disclosure.

The application claims benefit of and incorporates by reference in its entirety U.S. Provisional Application No. 61/125,967, filed Apr. 30, 2008.

1. A method of degrading pyrethroid, the method comprising contacting pyrethroid with a composition comprising the enzyme organophosphate hydrolase (OPH).

2. The method according to claim 1, wherein the pyrethroid is present in a sample, the method comprising contacting the sample with a composition comprising the enzyme organophosphate hydrolase (OPH).

3. The method according to claim 1, wherein the pyrethroid is completely degraded or at least partially degraded.

4. The method according to claim 1, wherein the pyrethroid is selected from the group consisting of acrinathrin, alpha-cypermethrin, betacyfluthrin, cyhalothrin, cypermethrin, deltamethrin, esfenvalerate, ethofenprox, fenpropathrin, fenvalerate, flucythrinate, lambda-cyhalothrin, permethrin, taufluvalinate, fluvalinate, tralomethrin, zetacypermethrin, cyfluthrin, bifenthrin, cycloprothrin, eflusilanate, fubfenprox, pyrethrin, resmethrin, gamma-cyhalothrin, allethrin, tetramethrin, d-phenothrin, bioresmethrin, cyphenothrin and sumithrin.

5. The method according to claim 1, wherein the pyrethroid is selected from the group consisting of allethrin, tetramethrin, resmethrin, d-phenothrin, bioresmethrin, and permethrin.

6. The method according to claim 1, wherein the pyrethroid is selected from the group consisting of cypermethrin, cyfluthrin, deltamethrin, cyphenothrin, fenvalerate, and fluvalinate.

7. The method according to claim 1, wherein the pyrethroid is selected from the group consisting of permethrin, resmethrin, and sumithrin.

8. The method according to claim 1, wherein the pyrethroid is permethrin.

9. The method according to claim 1, wherein said sample has a pH in the range of about 6.5 to about 9.0.

10. The method according to claim 2, wherein the pH is about 8.5.

11. The method of claim 10, wherein the pH of the sample is adjusted to 8.5.

12. The method according to claim 2, wherein the temperature of the sample is in the range of about 20° C.-30° C.

13. The method of claim 12, wherein the temperature of the sample is about 22° C.

14. The method according to claim 1, wherein the sample is effluent from a manufacturing process.

15. The method of claim 14, wherein the manufacturing process comprises the treatment of carpeting with pyrethroid.

16. A method of degrading pyrethroid in a sample, the method comprising contacting the sample with a composition comprising a microbe that expresses OPH.

17. The method of claim 16, wherein the microbe is a bacterium.

18. A method of inactivating pyrethroid, the method comprising contacting the pyrethroid with a composition comprising OPH.

19. A method of rendering pyrethroid less active, the method comprising contacting the pyrethroid with a composition comprising OPH.

20. A method of inactivating pyrethroid in a sample, the method comprising contacting the sample with a composition comprising OPH.

21. A method of rendering pyrethroid less active in a sample, the method comprising contacting the sample with a composition comprising OPH.

22. The method according to claim 15, wherein the pyrethroid is permethrin.

23. A pyrethroid-degrading composition, the composition comprising at least one isolated enzyme.

24. The composition of claim 22, comprising OPH.

25. The composition according to claim 23, wherein the pyrethroid is permethrin.

26. Use of OPH for degrading pyrethroid.

27. The use according to claim 26, wherein the pyrethroid is permethrin.
